(19) United States
(12) Patent Application Publication
(10) Pub. No.: US 2024/0050483 A1
(43) Pub. Date: Feb. 15, 2024

(54) METHODS FOR GENERATING HEMATOPOIETIC STEM CELLS AND COMPOSITIONS THEREOF

(71) Applicant: GARUDA THERAPEUTICS, INC., Cambridge, MA (US)

(72) Inventor: Dhvanit SHAH, Chestnut Hill, MA (US)

(21) Appl. No.: 18/266,435

(22) PCT Filed: Dec. 10, 2021
(86) PCT No.: PCT/US21/62884

§ 371 Date: Jun. 9, 2023

Related U.S. Application Data
(60) Provisional application No. 63/123,778, filed on Dec. 10, 2020.

(51) Int. Cl. A61K 35/28; C12N 5/071; C12N 5/0789; C12N 15/86; A61K 39/00

(52) U.S. Cl. CPC A61K 35/28; C12N 5/069; C12N 5/0647; C12N 15/86; A61K 39/4611; A61K 39/4612; A61K 39/4613; A61K 39/4632; A61K 39/4631; C12N 2506/45; C12N 2710/10043

(57) ABSTRACT

In various aspects and embodiments, this disclosure provides methods for generating hematopoietic stem cells (HSCs), as well as compositions comprising the same, and methods of treating disease. The disclosure provides methods for preparing endothelial cells from pluripotent stem cells by expression (e.g., overexpression) of E26 transformation-specific variant 2 (ETV2) transcription factor. HSCs are then generated from the endothelial cells using mechanical, biochemical, pharmacological and/or genetic stimulation.

### CROSS-REFERENCE TO RELATED APPLICATIONS

This application claims the benefit of and priority to U.S. Provisional Patent Application No. 63/123,778, filed Dec. 10, 2020, the contents of which are hereby incorporated by reference in their entirety.

### BACKGROUND

Hematopoietic Stem Cell (HSC) transplantation (HSCT) involves the infusion of autologous or allogeneic stem cells to reestablish hematopoietic function in patients whose bone marrow or immune system is defective, such as in therapy for hematological cancers. HSCs are also useful for producing cancer immunotherapies, including by the introduction of genes encoding T-cell receptors (TCR) or chimeric antigen receptors (CARs) directed against tumor-associated antigens. Specifically, cells that engraft in the subject for long-term blood cell production could provide a long-term source of targeted anti-cancer effector cells to sustain remissions. Gschweng et al., Hematopoietic stem cells for cancer immunotherapy, Immunol Rev. 2014 257(1):237-49.

However, ex vivo HSC expansion remains a significant hurdle, and particularly for deriving HSC products that can effectively engraft upon administration to the patient. Tajer P, et al., Ex Vivo Expansion of Hematopoietic Stem Cells for Therapeutic Purposes: Lessons from Development and the Niche, Cells 2019 February; 8(2): 169.

In various aspects and embodiments, the invention meets these objectives.

### BRIEF DESCRIPTION OF DRAWINGS

FIG. 1 shows that ETV2 over-expression (OE) does not affect pluripotency. FIG. 1 shows FACS plots representative of transduction efficiency of iPSC with an adenoviral vector to overexpress the ETV2 and the GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a-CD34+CD31+) and relative quantification demonstrates that ETV2-OE enhances the formation of hemogenic endothelial cells.

FIG. 3 shows that ETV2 over-expression (OE) enhances the CD34+ cell formation during iPSC differentiation. Representative flow cytometric analysis of CD34+ cells and relative quantification demonstrates that ETV2-OE enhances the CD34+ cell formation.

### DESCRIPTION OF THE INVENTION

In various aspects and embodiments, this disclosure provides methods for generating hematopoietic stem cells (HSCs), as well as compositions comprising the same, and methods of treating disease. HSCs produced according to the disclosure, in various embodiments, have advantages in establishing hematopoietic function and/or engraftment for long-term production of blood cell lineages, and/or for ex vivo production of cells for immunotherapy.

In some embodiments, this disclosure provides a method for generating hematopoietic stem cells (HSCs). The method comprises preparing endothelial cells from pluripotent stem cells by expression (e.g., overexpression) of E26 transformation-specific variant 2 (ETV2) transcription factor. HSCs are then generated from the endothelial cells using mechanical, biochemical, pharmacological and/or genetic stimulation. A nucleotide sequence encoding ETV2 is described in Wang K, et al., Robust differentiation of human pluripotent stem cells into endothelial cells via temporal modulation of ETV2 with mRNA. Sci. Adv. Vol. 6 (2020), which is hereby incorporated by reference in its entirety.

In accordance with embodiments of this disclosure, ETV2 overexpression in iPSCs does not affect their pluripotency properties and facilitates their ability to undergo the hemogenic endothelial and hematopoietic differentiation.

In some embodiments, the pluripotent stem cells are induced pluripotent stem cells (iPSCs) prepared by reprogramming somatic cells. For example, somatic cells may be reprogrammed by expression of reprogramming factors selected from one or a combination selected from Sox2, Oct3/4, c-Myc, Nanog, Lin28, and Klf4. In some embodiments, the reprogramming factors are Sox2, Oct3/4, c-Myc, Nanog, Lin28, and Klf4. In some embodiments, the reprogramming factors are Sox2, Oct3/4, c-Myc, and Klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well-known viral vector systems, such as lentiviral or Sendai viral systems. Alternatively, reprogramming factors may be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations. In some embodiments, iPSCs are generated from somatic cells such as (but not limited to) fibroblasts or PBMCs.

In some embodiments, the iPSCs are derived from T-cells, B-cells, cord blood (including from CD3+ or CD8+ cells from cord blood), CD34+ cells or CD34-enriched cells, or other human primary tissues. In various embodiments, the iPSCs are autologous or allogenic (e.g., HLA-matched) with respect to a recipient. In some embodiments, iPSCs are HLA-modified or HLA-null cells. In these or other embodiments, the iPSCs are gene-modified and may comprise genes for expression of a chimeric antigen receptor (CAR) or CCR5.

In some embodiments, the iPSCs comprise one or more constitutive or inducible suicide gene(s). In these embodiments, after treatment with the HSCs, transplanted HSCs can be subject to easy depletion. In exemplary suicide gene is HSV-thymidine kinase (HSV-TK), as described in Liang Q, et al., Linking a cell-division gene and a suicide gene to define and improve cell therapy safety. Nature Vol. 563: 701-704 (2018). HSV-TK converts ganciclovir into a toxic product and thus allows for selective elimination of TK+ cells using this active agent. An alternative or additional suicide gene is Caspase-9. Yagyu S. et al., An Inducible Caspase-9 Suicide Gene to Improve the Safety of Therapy Using Human Induced Pluripotent Stem Cells. Mol. Ther. 2015 23(9):1475-85. See also, Wiebking V., et al., Metabolic engineering generates a transgene-free safety switch for cell therapy. Nature Biotechnology Vol. 38: 1441-1450 (2020).

In these or other embodiments, the iPSCs may comprise a reporter gene, allowing for detection of the HSCs prepared according to the process, including after administration to a patient. Exemplary reporter genes include Positron Emission Tomography (PET) imaging reporter genes, which allow for whole-body monitoring of, for example, therapeutic cell locations, quantity at all locations, survival and proliferation over time. Exemplary PET reporting genes are described, for example, in Yaghoubi S. et al., Positron Emission Tomography Reporter Genes and Reporter Probes: Gene and Cell Therapy Applications. Theranostics. 2012; 2(4): 374-391. An exemplary PET reporter gene is PMK2. See, Haywood T., et al., Positron emission tomography reporter gene strategy for use in the central nervous system. PNAS 2019 116(23): 11402-11407.

In various embodiments, the iPSCs are differentiated to mesodermal progenitor cells (MPCs), and the MPCs are differentiated to the endothelial cells (ECs). Differentiation to MPCs is generally through activation of Wnt and Nodal signaling pathways. See Patsch C., et al., Generation of vascular endothelial and smooth muscle cells from human pluripotent stem cells. Nat. Cell. Biol. 17, 994-1003 (2015). For example, Wnt and Nodal signaling pathways can be activated using a glycogen synthase kinase 3 inhibitor (e.g., CHIR99021). See, Wang K, et al., Robust differentiation of human pluripotent stem cells into endothelial cells via temporal modulation of ETV2 with mRNA. Sci. Adv. Vol. 6 (2020). ETV2 expression in MPC cells is involved in the differentiation to ECs. A heterologous ETV2 can be expressed, for example, by introduction of an encoding non-integrating episomal plasmid, for constitutive or inducible expression of ETV2, and for production of transgene-free ECs. In some embodiments, ETV2 is expressed from an mRNA introduced into the MPCs. mRNA can be introduced using any available method, including electroporation or lipofection. Differentiation of MPCs expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). In some embodiments, expression of ETV2 can employ a viral vector.

In some embodiments, iPSCs (e.g., expressing ETV2) are used to generate embryoid bodies (EB), which can be used for inducing EHT. Preparation of EBs is described, for example, in US 2019/0177695, which is hereby incorporated by reference in its entirety. In some embodiments, human iPSC aggregates are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93.

In some embodiments, a population of human pluripotent stem cells that have been induced to undergo differentiation (e.g., to endothelial cells), are enriched, for example, as described in U.S. Pat. No. 9,834,754, which is hereby incorporated by reference in its entirety. For example, this process can comprise inducing differentiation in a population of human pluripotent stem cells (or EBs derived therefrom), and sorting the induced population based on expression of CD34 and/or CD43. A fraction is selected that is CD34+ and/or CD43^{neg}.

The ECs produced by this process can be transitioned to hemogenic endothelial cells (HECs) or HSCs using mechanical, biochemical, genetic, and/or pharmacological stimulation. In some embodiments, the method comprises increasing activity or expression of DNA (cytosine-5-)-methyltransferase 3 beta (Dnmt3b) and/or GTPase IMAP Family Member 6 (Gimap6) in the endothelial cells (e.g., using mechanical, pharmacological, or genetic means) under conditions sufficient for stimulating formation of HSCs. See WO 2019/236943, which is hereby incorporated by reference in its entirety. In some embodiments, the ECs are transitioned to hemogenic endothelial (HE) cells using the mechanical, genetic, and/or pharmacologic stimulation, which can then be transitioned to HSCs, optionally using the mechanical, genetic, and/or pharmacologic stimulation.

In some embodiments, the pharmacological stimulation involves contacting the endothelial cells with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yodal. Yodal (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015). Yoda 1 has the following structure:

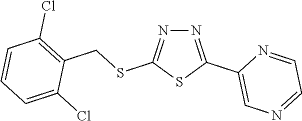 [SMILES: Clc1cccc(Cl)c1CSc1nnc(-c2cnccn2)s1]

Derivatives of Yodal can be employed in various embodiments. For example, derivatives comprising a 2,6-dichlorophenyl core are employed in some embodiments. Exemplary agonists are disclosed in Evans EL, et al., Yodal analogue (Dookul) which antagonizes Yodal-evoked activation of Piezol and aortic relaxation, British J. of Pharmacology 175(1744-1759): 2018. Still other Piezol agonist include Jedi1, and/or Jedi2, or a derivatives thereof. See Wang Y., et al., A lever-like transduction pathway for long-distance chemical- and mechano-gating of the mechanosensitive Piezol channel. Nature Communications (2018) 9:1300. Jedi1 and Jedi2 have a 3-carboxylic acid methylfuran structural motif Other Piezol agonists sharing this motif may be employed in accordance with embodiments of the invention. These Piezol agonists are commercially available. In various embodiments, the effective amount of the Piezol agonist or derivative is in the range of about 0.1 μM to about 500 μM, or about 0.1 μM to about 200 μM, or about 0.1 μM to about 100 μM, or in some embodiments, in the range of about 1 μM to about 150 μM, or about 5 μM to about 100 μM, or about 10 μM to about 50 μM, or about 20 μM to about 50 μM.

Alternatively or in addition, transition to HE cells or HSCs is by genetic stimulus. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the endothelial cells, such as to increase promoter strength, ribosome binding, RNA stability, or impact RNA splicing.

In some embodiments, the method comprises increasing the activity or expression of Gimap6 in the endothelial cells, alone or in combination with Dnmt3b. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the endothelial cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents known in the art. In some embodiments, expression of Dnmt3b and/or Gimap6 is increased by a transgene-free method (e.g., episome delivery as described).

In some embodiments, expression or activity of Dnmt3b and/or Gimap6 or other genes disclosed herein are increased using a gene editing technology, for example, to introduce one or more modifications to increase promoter strength, ribosome binding, or RNA stability. Various editing technologies are known, and include CRISPR, zinc fingers (ZFs) and transcription activator-like effectors (TALEs). Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducting using CRISPR associated Cas system, as known in the art. See, for example, U.S. Pat. Nos. 8,697,359, 8,906,616, and 8,999,641, which is hereby incorporated by reference in its entirety.

In some embodiments, the ECs or HECs are transitioned to HSCs using mechanical stimulus, such as a process comprising circumferential stretch in 2D or 3D culture. For example, a cell population comprising developmentally plastic endothelial cells or HE cells is introduced to a bioreactor. In some embodiments, the bioreactor provides a cyclic-strain biomechanical stretching, as described in WO 2017/096215, which is hereby incorporated by reference in its entirety. The cyclic-strain biomechanical stretching increases the activity or expression of Dnmt3b and/or Gimap6. In these embodiments, mechanical means apply stretching forces to the cells. For example, a computer controlled vacuum pump system (e.g., the FlexCell™ Tension System, the Cytostretcher System, or similar) attached to a nylon or similar biocompatible or biomimetic membrane of a flexible-bottomed culture plate can be used to apply circumferential stretch ex vivo to cells under defined and controlled cyclic strain conditions.

In some embodiments, endothelial cells of HE cells are sorted or enriched for cells having a desired cell surface marker phenotype. In some embodiments, the endothelial cells or HE cells are selected or enriched for CD34+ cells. Optionally, the cells are sorted or enriched based on one or more of the following cell surface markers: CD31^{pos}, CD144^{pos}, KDR^{pos}, CD43^{neg}, and CD235^{neg}.

In some embodiments, the method comprises, at least in part, inducing hematopoietic differentiation by culturing endothelial cells or HE cells with one or more of factors selected from Insulin Growth Factor-1 (IGF-1), Sonic Hedgehog (SHH), Angiotensin 2, Losartan, Flt3, Flt3-ligand, Y27632, FGF, FGF-2(bFGF), BMP-4, Activin A, Transferrin, VEGF, DKK, IL-6, IL011, SCF, EPO, IL-3, SB-431542, Fibronectin, and Vitronectin.

In various embodiments, the HSCs generated are expanded. For example, the HSCs can be expanded according to methods disclosed in U.S. Pat. Nos. 8,168,428; 9,028,811; 10,272,110; and 10,278,990, which are hereby incorporated by reference in their entireties. For example, in some embodiments, ex vivo expansion of HSCs employs addition of prostaglandin E₂ (PGE₂) or a PGE₂ derivative or precursor to the culture. In these or other embodiments, ex vivo expansion employs addition of linoleic acid to the culture. In these or other embodiments, the HSC expansion employs addition of polyvinyl alcohol to the culture. In some embodiments, HSC expansion comprises, at least in-part, culturing the cells with aryl hydrocarbon receptor antagonists, such as substituted imidazopyridines and imidazopyrazines, including those described in U.S. Pat. No. 10,457,683, which is hereby incorporated by reference in its entirety. Other compounds and reagents useful for expanding HSCs are described in Papa L., et al., Distinct Mechanisms Underlying the Ex Vivo Expansion of Human Cord Blood Stem Cells with Different Strategies Currently Used for Allogeneic Transplantation, Blood(2019) 134 (Supplement 1): 4470. Such compounds and reagents can include nicotinamide and valproic acid.

In various embodiments, the hematopoietic stem cells produced according to this disclosure comprise long term hematopoietic stem cells (LT-HSCs), which exhibit superior engraftment, and reconstitute to functional, multi-lineage adult blood in the recipient. In some embodiments, HSCs include Lin−/Scal+/c-kit+ cells. LT-HSCs self-renew to sustain the stem cell pool or differentiate into short-term HSCs (ST-HSCs) or lineage-restricted progenitors that undergo extensive proliferation and differentiation to produce terminally differentiated, functional hematopoietic cells. ST-HSCs or multipotent progenitors (MPPs) are only able to sustain hematopoiesis in the short term, while the LT-HSCs can persist for the lifespan of the organism to perpetually replenish the hematopoietic system. In some embodiments of this disclosure, the HSCs comprise at least about 0.0001% HSCs, or at least about 0.001% LT-HSCs, or at least about 0.01% LT-HSCs, or at least about 0.1% LT-HSCs, or at least about 1% LT-HSCs. In some embodiments, subpopulations of cells (e.g., LT-HSCs) can be isolated or enriched using, for example, cell sorting approaches. For example, in some embodiments, cells are selected or enriched for CD34+ cells. In some embodiments, cells are enriched or sorted based on the expression of one or more of CD34, CD45, CD38, CD90, CD49f, and GPI-80. In some embodiments, cells are enriched or sorted for cells having one or more of the following phenotypic markers: CD110+, CD135+, and APLNR+.

In various embodiments, during transition to HSCs, cells are recovered that are suspended in the culture (e.g., non-adherent).

In some aspects, this disclosure provides a composition comprising an HSC population made according to the method described herein. In various embodiments, a composition for cellular therapy is prepared that comprises a population of HSCs prepared by the methods described herein, and a pharmaceutically acceptable vehicle. The pharmaceutical composition may comprise at least about 10² HSCs, or at least about 10³ HSCs, or at least about 10⁴ HSCs, or at least about 10⁵ HSCs, or at least about 10⁶ HSCs, or at least about 10⁷ HSCs, or at least 10⁸ HSCs. For example, in some embodiments, the pharmaceutical composition is administered, comprising from about 100,000 to about 400,000 (CD34+) HSCs per kilogram (e.g., about 200,000 cells/kg) of a recipient's body weight.

The HSCs for therapy or transplantation can be generated in some embodiments in a relatively short period of time, such as less than two months, or less than one month, or less than about two weeks.

The cell composition may further comprise a pharmaceutically acceptable carrier or vehicle suitable for intravenous infusion or other administration route, and may include a suitable cryoprotectant. An exemplary carrier is DMSO (e.g., about 10% DMSO). Cell compositions may be provided in unit vials or bags, and stored frozen until use. In certain embodiments, the volume of the composition is from about one fluid ounce to one pint.

HSCs generated using the methods described herein are administered to a subject (a recipient), e.g., by intravenous infusion or intra-bone marrow transplantation. The methods can be performed following myeloablative, non-myeloablative, or immunotoxin-based (e.g. anti-c-Kit, anti-CD45, etc.) conditioning regimes.

The methods described herein can be used to generate populations of HSC for use in transplantation protocols, e.g., to treat blood (malignant and non-malignant), bone marrow, metabolic, and immune diseases. In some embodiments, the HSC populations are derived from autologous cells or universally-compatible donor cells or HLA-modified or HLA null cells. In some embodiments, the HSC population is derived from gene-edited cells, comprising one or more transgenes (e.g., CCR5, TCR, or CAR). That is, HSC populations are generated from iPSCs that were prepared from cells of the recipient subject or prepared from donor cells (e.g., universal donor cells, HLA-matched cells, HLA-modified cells, or HLA-null cells), and which are optionally gene edited.

In some embodiments, the recipient subject has a condition selected from acute myeloid leukemia; acute lymphoblastic leukemia; chronic myeloid leukemia; chronic lymphocytic leukemia; myeloproliferative disorders; myelodysplastic syndromes; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); acquired immune deficiency syndrome (AIDS); Wiskott-Aldrich syndrome; hemophagocytic lymphohistiocytosis; inborn errors of metabolism; epidermolysis bullosa; severe congenital neutropenia; Shwachman-Diamond syndrome; Diamond-Blackfan anemia; and leukocyte adhesion deficiency.

In still other embodiments, the HSCs produced according to this disclosure are used to produce immune cells for immune therapy, such as T cells (e.g., heterologous TCR-expressing T cells, chimeric antigen receptor T cells (CAR-T cells), T-regulatory cells, cytotoxic T cells, gamma-delta T cells, alpha-beta T cells, etc.) NK cells, (including CAR-NK cells) or B-cells (including CAR-B cells).

In other embodiments, the HSCs produced according to this disclosure are used to produce macrophages for treating graft tolerance, autoimmune disorders, or fibrosis, for example.

In some embodiments, the HSCs produced according to this disclosure are used to produce red blood cells, which can be used to treat, for example, hemoglobinopathies. In still other embodiments, the HSCs produced according to this disclosure are used to produce platelets for treatment of direct or indirect bleeding disorders. In some embodiments, the HSCs are used to prepare blood products for treating co-morbidities associated with blood, bone marrow, immune, metabolic, or mitochondrial disorders.

Methods for differentiation to desired cell lineages are known in the art.

As used herein, the term “about” means±10% of the associated numerical value.

### EXAMPLES

### Example 1 ETV2 Over-Expression Increases the Yield of Hemogenic Endothelial Cells and Enhances the CD34+ Cell Formulation During iPSC Differentiation but does not Affect Pluripotency

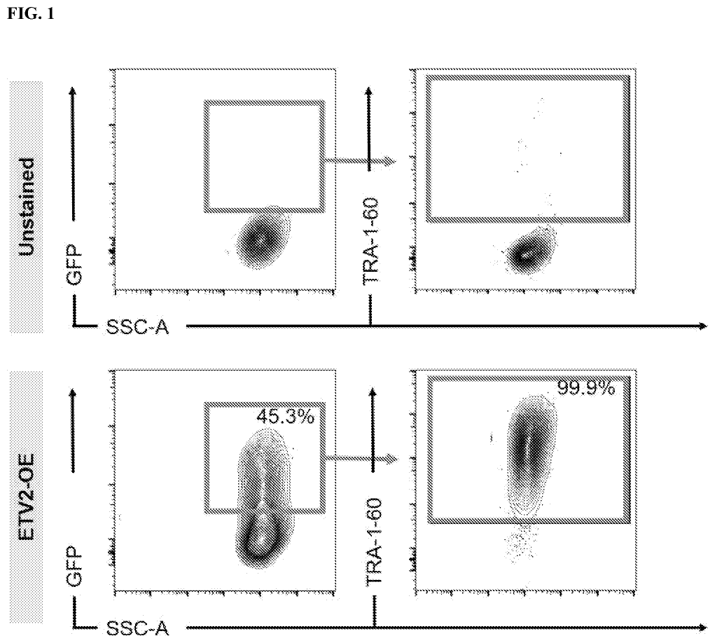

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the stemness marker expression TRA-1-60 (FIG. 1). FIG. 1 shows FACS plots representative of transduction efficiency of iPSC with an adenoviral vector to overexpress the ETV2 and the GFP sequences.

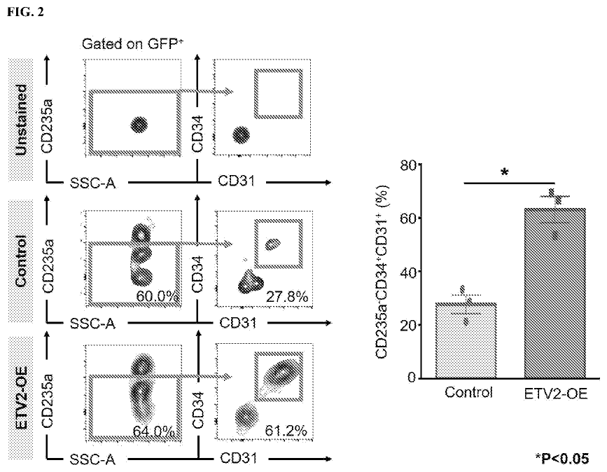

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34+ and CD31+ markers within the CD235a-population (FIG. 2). Specifically, FIG. 2 shows representative flow cytometric analysis of hemogenic endothelial cells (defined here as CD235a-CD34+CD31+) and relative quantification demonstrates that ETV2-OE enhances the formation of hemogenic endothelial cells.

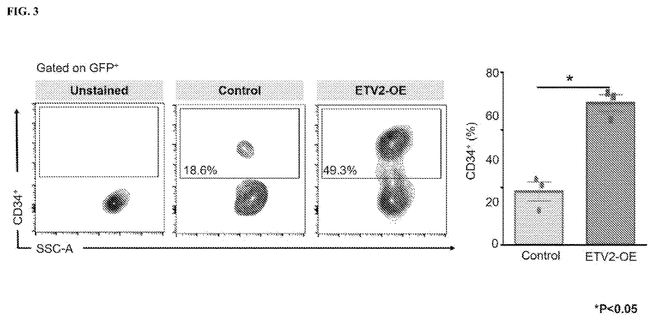

Moreover, the results suggest that ETV2-OE enhances the formation of the CD34+ cells (FIG. 3). FIG. 3 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiation.

1. A method for generating hematopoietic stem cells (HSCs), comprising:
preparing endothelial cells from pluripotent stem cells by expression of E26 transformation-specific variant 2 (ETV2) transcription factor;
transitioning the endothelial cells to hemogenic endothelial cells, and transitioning the hemogenic endothelial cells to hematopoietic stem cells.

2. The method of claim 1, wherein the pluripotent stem cells are induced pluripotent stem cells (iPSCs) prepared by reprogramming somatic cells.

3. The method of claim 2, wherein the somatic cells are reprogrammed by expression of reprogramming factors selected from one or more of Sox2, Oct3/4, c-Myc, Nanog, Lin28, and Klf4.

4. The method of claim 3, wherein the iPSCs are autologous or allogenic with respect to a recipient.

5. The method of any one of claims 1 to 4, wherein iPSCs are HLA-modified or HLA-null cells.

6. The method of any one of claims 1 to 5, wherein the iPSCs are gene modified.

7. The method of any one of claims 1 to 6, wherein the iPSCs comprise a constitutive or inducible suicide gene.

8. The method of any one of claims 1 to 7, wherein the iPSCs comprise a reporter gene.

9. The method of any one of claims 1 to 8, wherein ETV2 is expressed from a non-integrating episome.

10. The method of claim 9, wherein ETV2 expression is constitutive or inducible.

11. The method of any one of claims 1 to 8, wherein ETV2 is expressed from an mRNA introduced into the iPSCs.

12. The method of claim 11, wherein ETV2 mRNA is introduced to mesodermal progenitor cells (MPCs) prepared from the iPSCs.

13. The method of any one of claims 1 to 12, wherein the iPSCs are differentiated to mesodermal progenitor cells (MPCs), and the MPCs are differentiated to the endothelial cells (ECs).

14. The method of claim 13, wherein MPCs are differentiated to ECs by addition of VEGF-A.

15. The method of claim 13 or 14, wherein ECs are differentiated to hemogenic endothelial cells (HECs) by addition of a Piezol agonist.

16. The method of claim 15, wherein the Piezol agonist is Yodal, Jedi1, and/or Jedi2.

17. The method of any one of claims 13 to 16, wherein ECs are differentiated to HECs by applying cyclic stretch.

18. The method of any one of claims 1 to 16, further comprising selecting or enriching for cells that are CD34^{pos}, and optionally having a cell surface phenotype selected from one or more of: CD31^{pos}, CD144^{pos}, KDR^{pos}, CD235^{neg}, and CD43^{neg}.

19. The method of any one of claims 1 to 18, wherein HECs are differentiated to HSCs by addition of a Piezol agonist.

20. The method of claim 19, wherein the Piezol agonist is Yodal, Jedi1, and/or Jedi2.

21. The method of claim 19 or 20, wherein HECs are differentiated to HSCs by applying cyclic stretch.

22. The method of any one of claims 1 to 21, wherein inducing hematopoietic differentiation comprises or further comprises culturing endothelial cells or HE cells with one or more of Insulin Growth Factor-1 (IGF-1), Sonic Hedgehog (SHH), Angiotensin 2, Losartan, Flt3, Flt3-ligand, Y27632, FGF, FGF-2(bFGF), BMP-4, Activin A, Transferrin, VEGF, DKK, IL-6, IL011, SCF, EPO, TPO, IL-3, SB-431542, Fibronectin, and Vitronectin.

23. The method of any one of claims 1 to 22, wherein the ECs are transitioned to HECs or HSCs by a process comprising increasing expression or activity of Dnmt3b and/or Gimap6.

24. The method of claim 23, wherein a Dnmt3b and/or Gimap6 gene is expressed from an episome.

25. The method of claim 22, wherein Dnmt3b and/or Gimap6 is expressed from an mRNA introduced into the iPSCs, ECs or a precursor thereof.

26. The method of any one of claims 1 to 25, wherein the HSCs are expanded, optionally with linoleic acid, PGE₂, PGE₂ derivative, and/or PGE₂ precursor(s).

27. The method of any one of claims 1 to 26, wherein HSC expansion comprises culturing the cells with a compound described in U.S. Pat. No. 10,457,683, which is hereby incorporated by reference.

28. The method of any one of claims 1 to 26, wherein HSC expansion comprises culturing the cells with one or more of polyvinyl alcohol, UM171, nicotinamide, and valproic acid.

29. The method of any one of claims 1 to 28, wherein the HSCs comprise Long Term-HSCs (LT-HSCs).

30. The method of claim 29, further comprising selecting or enriching for CD34+ cells, and optionally sorting or enriching cells based on the positive or negative expression of one or more of CD45, CD38, CD90, CD49f, and GPI-80.

31. The method of claim 28 or 29, comprising, recovering cells suspended in the culture.

32. The method of any one of claims 1 to 31, wherein the HSCs comprise at least 0.0001% LT-HSCs.

33. A composition comprising an HSC population made according to the method of any one of claims 1 to 32.

34. A method for treating a disease or condition in a recipient, comprising, administering the composition of claim 33 to said recipient.

35. The method of claim 34, wherein the disease or condition is a blood, bone marrow, metabolic, immune or mitochondrial disease.

36. The method of claim 35, wherein the disease or condition is selected from acute myeloid leukemia; acute lymphoblastic leukemia; chronic myeloid leukemia; chronic lymphocytic leukemia; myeloproliferative disorders; myelodysplastic syndromes; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); acquired immune deficiency syndrome (AIDS); Wiskott-Aldrich syndrome; hemophagocytic lymphohistiocytosis; inborn errors of metabolism; epidermolysis bullosa; severe congenital neutropenia; Shwachman-Diamond syndrome; Diamond-Blackfan anemia; and leukocyte adhesion deficiency.

37. The method of any one of claims 34 to 36, wherein the HSCs are derived from allogeneic-derived or universally-compatible donor cells, HLA-modified or HLA-null cells; and are optionally gene modified.

38. The method of any one of claims 34 to 37, wherein the HSCs are derived from cells of the recipient.

39. A method for preparing a cell population for immune therapy, comprising differentiating HSCs produced according to any one of claims 1 to 32, to an immune cell lineage.

40. The method of claim 39, wherein the immune cell lineage is a T cell, B cell, or NK cell.

41. The method of claim 40, wherein the immune cell lineage is a T-regulatory cell, cytotoxic T cell, gamma-delta T cell, or alpha-beta T cell.

42. The method of claim 41, further comprising, expressing a heterologous TCR.

43. The method of claim 40 or 41, further comprising, expressing a chimeric antigen receptor (CAR).

44. A method for preparing a cell population for treating hemoglobinopathies, comprising, differentiating HSCs produced according to any one of claims 1 to 32 to red blood cells.

45. A method for preparing a cell population for treating graft tolerance, an autoimmune disorder, or fibrosis, comprising differentiating HSCs produced according to any one of claims 1 to 32, to macrophages.

46. A method for preparing a cell population for directly or indirectly treating a bleeding disorder, comprising differentiating HSCs produced according to any one of claims 1 to 32, to platelets.
